(19) United States
(12) Patent Application Publication
(10) Pub. No.: US 2019/0066843 A1
(43) Pub. Date: Feb. 28, 2019

(54) COLLAPSING CLINICAL EVENT DATA INTO MEANINGFUL STATES OF PATIENT CARE

(71) Applicant: KONINKLIJKE PHILIPS N.V., EINDHOVEN (NL)

(72) Inventor: Eric Thomas Carlson, New York, NY (US)

(21) Appl. No.: 16/100,937

(22) Filed: Aug. 10, 2018

Related U.S. Application Data
(60) Provisional application No. 62/548,478, filed on Aug. 22, 2017.

(51) Int. Cl. G16H 50/20; G16H 50/70

(52) U.S. Cl. CPC G16H 50/20; G16H 50/70

(57) ABSTRACT

Techniques are described herein for collapsing clinical event data into meaningful states of patient care. In various embodiments, time-ordered streams of clinical data associated with a plurality of respective patients may be divided into one or more respective pluralities of temporal segments. Each stream of clinical data may indicate a clinical history of a particular patient of the plurality of patients. Each of the one or more pluralities of temporal segments may have a different duration. In some embodiments, embedding(s) of the one or more pluralities of temporal segments into reduced dimensionality space(s) may be generated. Process mining may be performed on the embedding(s). Based on the process mining, one or more temporal health trajectories shared among the plurality of patients may be identified.

### CROSS-REFERENCE TO RELATED APPLICATIONS

This application claims priority to U.S. provisional patent application No. 62/548,478, filed on Aug. 22, 2017, the entire disclosure of which is hereby incorporated by reference for all purposes.

### TECHNICAL FIELD

Various embodiments described herein are directed generally to artificial intelligence. More particularly, but not exclusively, various methods and apparatus disclosed herein relate to collapsing clinical event data into meaningful states of patient care.

### BACKGROUND

Diagnosis of a clinical condition is a challenging task, which often requires significant medical investigation. Clinicians perform complex cognitive processes to infer the probable diagnosis after observing several variables such as the patient's past medical history, current condition, and various clinical measurements. The cognitive burden of dealing with complex patient situations could be reduced by automatically generating and providing information to physicians regarding current patient states, most probable diagnostic options for optimal clinical decision-making, and so forth.

Process mining may be used discover processes from data. Unfortunately, clinical data (e.g., hospital data) tends to be noisy. Similar patients can have numerous events (e.g., orders, lab tests, prescriptions, observations, notes, claims, measurements, medication, etc.) per day, often in different orders, and there is often extra or missing data. Moreover, patients may undergo “bursts” of relatively frequent clinical events in short time spans, but then may also experience longer time spans (e.g., recovery, physical therapy, outpatient care, etc.) with infrequent clinical events. All of this noise makes process mining difficult. Deep-learning approaches have the potential to create consistent, clean stages of care progression from this data, but tools derived for NLP do not cleanly apply to time-ordered (e.g., streaming) clinical event logs.

### SUMMARY

The present disclosure is directed to methods and apparatus for collapsing clinical event data into meaningful states of patient care. For example, multiple time-ordered streams of clinical data, which can include billing codes, lab results, treatments applied, clinical observations (e.g., free form notes in electronic health records, or “EHRs”), orders, etc., may indicate respective clinical histories of multiple patients. These streams may be divided into temporal segments of various durations. The durations of the segments may be selected based on a variety of criteria, such as whether enough patients share temporal segments such that patterns emerge. In some embodiments, the temporal segments may be embedded into a reduced dimensionality space. Resulting clusters of temporal segments may be examined to determine whether the clusters themselves are sufficient (e.g., include a threshold number of patients) and/or whether meaningful patterns—e.g., temporal health trajectories—emerge between clusters.

The temporal health trajectories may then be used for various purposes. One purpose may be determining, based on records/logs of a particular health care system, whether the particular health care system exhibits temporal health trajectories that are similar to, or diverge from, those of another health care system (or multiple health care systems generally), which may indicate suboptimal clinical procedures or policies. Another purpose may be determining a particular patient's state in a particular temporal health trajectory, so that potential next states (e.g., diagnoses, treatments, outcomes, etc.) may be predicted and treatment administered accordingly.

Generally, in one aspect, a method may include the following operations: dividing time-ordered streams of clinical data associated with a plurality of respective patients into one or more respective pluralities of temporal segments, wherein each stream of clinical data indicates a clinical history of a particular patient of the plurality of patients, and wherein each of the one or more pluralities of temporal segments has a different duration; generating one or more pluralities of embeddings of the one or more pluralities of temporal segments into a reduced dimensionality space; performing process mining on the one or more pluralities of embeddings; and based on the process mining, identifying one or more temporal health trajectories shared among the plurality of patients.

In various embodiments, the process mining may include: analyzing a first plurality of embeddings of the one or more pluralities of embeddings generated from a first plurality of temporal segments having a first duration to identify a first plurality of clusters of temporal segments in the reduced dimensionality space that share one or more attributes; determining that the first plurality of clusters of temporal segments in the reduced dimensionality space fail to satisfy a population criterion; analyzing a second plurality of embeddings of the one or more pluralities of embeddings generated from a second plurality of temporal segments having a second duration to identify a second plurality of clusters of temporal segments in the reduced dimensionality space that share one or more attributes; and determining that the second plurality of clusters of temporal segments in the reduced dimensionality space satisfy the population criterion. In various embodiments, the one or more temporal health trajectories may be identified based on the second plurality of clusters of temporal segments.

In various embodiments, the population criterion may be satisfied where a threshold number of patients are represented in each of a plurality of clusters. In various embodiments, the generating may include applying each of the one or more pluralities of temporal segments as input across a neural network to learn a respective one of the one or more pluralities of embeddings into the reduced dimensionality space. In various embodiments, the neural network may be a skip-gram model.

In various embodiments, each of the one or more pluralities of temporal segments may have a duration selected from an hour, a day, a week, or a month. In various embodiments, each of the one or more pluralities of embeddings may be represented as weights associated with a hidden layer of a neural network. In various embodiments, each temporal segment may include one or more clinical events that occurred during the temporal segment. In various embodiments, the one or more clinical events may be considered coincident within the temporal segment, regardless of an order in which the one or more clinical events actually occurred.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 schematically illustrates an example architecture and process flow that may be utilized in various embodiments described herein.

FIG. 2 depicts example neural network models in accordance with the prior art that may be used to perform selected aspects of the present disclosure.

FIG. 3 depicts an example temporal health trajectory that may be identified using techniques described herein.

FIG. 4 depicts an example method for practicing selected aspects of the present disclosure.

FIG. 5 depicts an example method for practicing selected aspects of the present disclosure.

FIG. 6 schematically depicts an example computer architecture.

### DETAILED DESCRIPTION

Diagnosis of a clinical condition is a challenging task, which often requires significant medical investigation. Clinicians perform complex cognitive processes to infer the probable diagnosis after observing several variables such as the patient's past medical history, current condition, and various clinical measurements. The cognitive burden of dealing with complex patient situations could be reduced by automatically generating and providing information to physicians regarding current patient states, most probable diagnostic options for optimal clinical decision-making, and so forth. Accordingly, techniques are described herein for collapsing clinical event data into meaningful states of patient care, e.g., so that what will be referred to herein as “temporal health trajectories” can be identified and used for various purposes.

In various embodiments, a patient's clinical history, which may include a plurality of clinical events (measurements, medication, notes, orders, labs, claims, etc.), may be organized into time-ordered streams of clinical data. These streams may be partitioned by durations of time into what will be referred to herein as “temporal segments.” Durations of these temporal segments may be varied (e.g., to minutes, hours, days, weeks, months, years, etc.) to set a scale of a window in which multiple clinical events are considered to be co-incident. In various embodiments, the durations of the temporal segments may be selected depending on disease pathway dynamics and other factors such as severity, acuity, etc. For instance, streams associated with patients in intensive care units (“ICU”) may be divided into shorter-duration temporal segments than patients suffering from chronic conditions. If temporal segment durations are set incorrectly—e.g., relatively short durations are used for patients suffering from chronic conditions that do not change often, or relatively long durations are used for ICU patients for which numerous clinical events occur at a relatively frequent pace—the disease states that emerge may be too narrow (i.e., match too few patients) or too broad (i.e., match too many patients).

Various process mining techniques may be employed, alone or in combination with other techniques described herein, to determine appropriate temporal segment durations and/or to identify temporal health trajectories. In some embodiments, a range of durations may be used to divide time-ordered streams of clinical data into temporal segments. Intra-temporal-segment event order may be discarded in some instances, such that all events within a temporal segment are considered co-incident. Process mining techniques may then be applied to the raw segmented data. In some embodiments, temporal segments may have durations that are optimized to ensure sufficient numbers of patients traverse various clinical temporal paths, while segregating patients sufficiently to prevent collapse of all patients to a single path (or too few paths).

In various embodiments, temporal segments may be embedded into reduced dimensionality space. These embeddings may be analyzed to identify clusters of similar temporal segments, as well as temporal health trajectories through multiple clusters. These temporal health trajectories may represent likely or possible disease or condition progressions that may be experienced by patients. In some embodiments, a so-called “skip-gram” algorithm (e.g., an algorithm employed by word2vec) may be applied to discover embeddings. The embeddings may be analyzed to collapse similar temporal segments into clusters based on distance (e.g., Kullback-Leibler, or “KL,” distance) in the reduced-dimensionality embedding space. Process mining may then be applied as described above, but based on these collapsed clusters rather than raw segments. In some embodiments, multiple embedding spaces, e.g., associated with multiple durations of temporal segments, may be considered. In some embodiments, a single embedding space with embeddings generated from temporal segments of multiple different durations may be considered. A temporal segment and/or embedding space may be chosen in some instances based on suitable temporal health trajectories emerging from that duration within that space.

In some embodiments, a variety of temporal segment durations may be used concurrently, e.g., with the same patient's data stream represented many times using different combinations of durations and time offsets. This may collapse multiple embedding spaces (e.g., each generated from a different temporal segment duration) into a single embedding space. Consequently, embeddings of differing durations and/or temporal offsets can nonetheless be related to each other, e.g., to identify temporal health trajectories. In some embodiments, a primary parameter in this method may be KL-distance to collapse points, which may in turn be optimized based on resulting pathways. In practical use for a single patient, any given time point for a patient will have many representative segments of differing durations. In some embodiments, a patient's effective current state may be derived as a geometric average in one or more of the aforementioned embedding spaces.

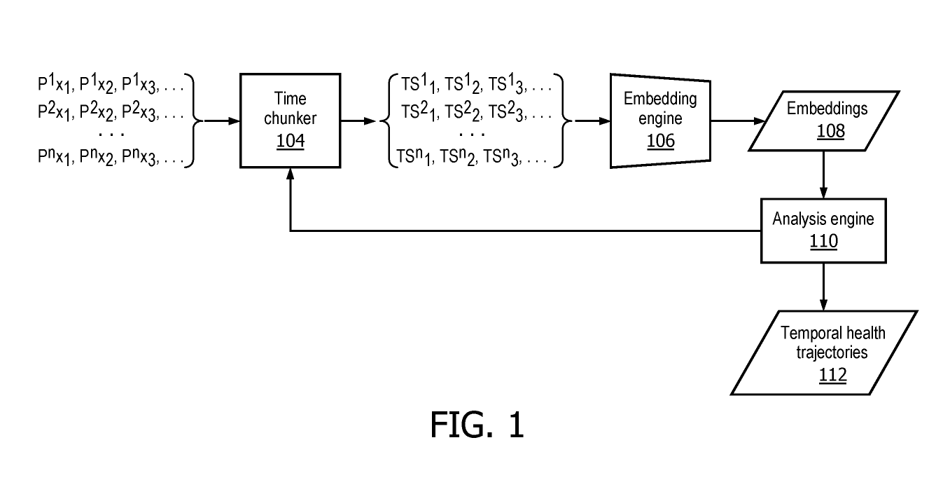

FIG. 1 schematically depicts one example of architecture and process flow that may be employed to practice selected aspects of the present disclosure. In FIG. 1, a plurality of time-ordered streams of clinical data, {(P¹x₁, P¹x₂, P¹x₃, . . . ), (P²x₁, P²x₂, P²x₃, . . . ), . . . , (Pⁿx₁, Pⁿx₂, Pⁿx₃, . . . )} associated with a number n of respective patients Pⁱ is provided as input. These time-ordered streams may indicate respective clinical histories of the patients. In various embodiments, each stream of clinical data may include a plurality of time-ordered clinical events x, such as lab results, observations (e.g., from clinician notes), symptoms, administered treatments, prescriptions, orders, measurements (e.g., blood pressure, heart rate, temperature, etc.), diagnoses, and so forth.

A frequency at which clinical events occur in a given stream of clinical data may depend on various factors, such as the patient's condition, the patient's treatment, physical therapy, and so forth. For example, a first stream associated with a first patient in an ICU may include a burst of numerous events that occurred/were observed during a relatively short period of time (e.g., multiple days, a week, a month, etc.) that the first patient was in ICU. Patient's experiencing relatively acute conditions such as acute renal failure, pregnancy, etc., may also exhibit burst(s) of frequent events. By contrast, a second stream associated with a second patient that suffers from a chronic condition (e.g., diabetes, heart disease, chronic kidney disease or “CKD,” etc.) may include clinical events at a lower frequency. Moreover, a stream associated with a single patient may include both periods of frequent clinical events (e.g., a hospital visit after an injury) and periods of less frequent clinical events (e.g., weeks or months of physical therapy following the hospital visit).

Accordingly, techniques are described herein for dividing, e.g., by a time chunker 104, the time-ordered streams of clinical data into one or more respective pluralities of temporal segments TS, such that {(TS¹₁, TS¹₂, TS¹₃, . . . ), (TS²₁, TS²₂, TS²₃, . . . ), . . . , (TSⁿ₁, TSⁿ₂, TSⁿ₃, . . . )}. In various embodiments, time chunker 104 may be implemented using any combination of hardware and/or software. In various embodiments, each plurality or set of temporal segments divided out by time chunker 104 may have a different duration, so that temporal segments of varying durations can be “tested” to determine which duration of temporal segments provides the best information (e.g., collapses into well-populated clusters in reduced dimensionality space, and/or with clear temporal health trajectories emerging between the clusters, etc.) that can be used for various purposes later.

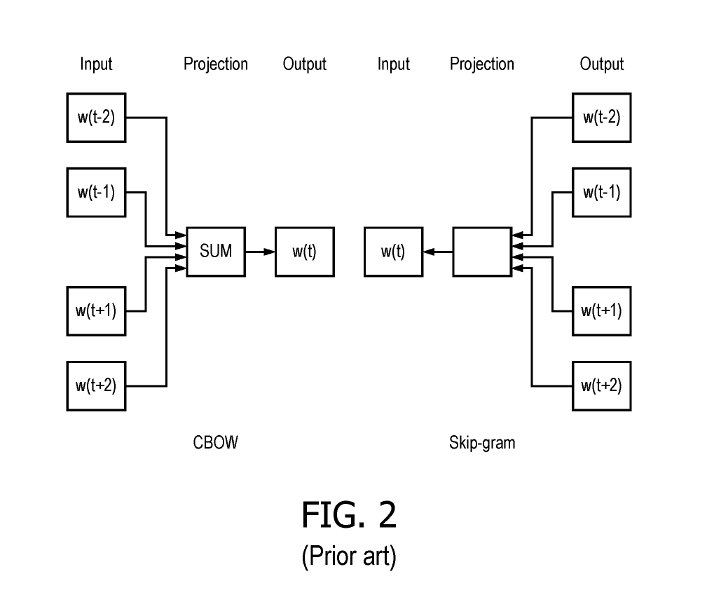

In some embodiments, the raw temporal segments may then be process mined to identify one or more temporal health trajectories. However, in other embodiments, an embedding engine 106 may be configured to generate one or more pluralities of embeddings 108 of the one or more pluralities of temporal segments {(TS¹₁, TS¹₂, TS¹₃, . . . ), (TS²₁, TS²₂, TS²₃, . . . ), . . . , (TSⁿ₁, TSⁿ₂, TSⁿ₃, . . . )} into a reduced dimensionality space. This embedding into reduced dimensionality space (or “feature extraction”) may be performed using various linear and/or nonlinear dimensionality reduction techniques, including but not limited to principal component analysis (“PCA”), linear discriminant analysis (“LDA”), multilinear subspace learning (for tensor representations), an so forth. In some embodiments, one or more neural networks may be used to learn embeddings. For example, FIG. 2 depicts a continuous bag-of-words (“CBOW”) neural network model and a skip-gram neural network that are used as part of the well-known “word2vec” group of related models and techniques. One or more of the models depicted in FIG. 2, especially the skip-gram model, may be used to learn embeddings of temporal segments into reduced dimensionality space, as will be described in more detail below.

Referring back to FIG. 1, in various embodiments, an analysis engine 110 may be configured to perform process mining on the one or more pluralities of embeddings 108 learned/generated by embedding engine 106. Based on the process mining, analysis engine 110 may identify one or more temporal health trajectories 112 shared among the plurality of patients associated with the original time-ordered streams of clinical data {(P¹x₁, P¹x₂, P¹x₃, . . . ), (P²x₁, P²x₂, P²x₃, . . . ), . . . , (Pⁿx₁, Pⁿx₂, Pⁿx₃, . . . )}.

Additionally or alternatively, in some embodiments, analysis engine 110 may be configured to determine, e.g., based on the process mining, whether various criteria are met by the one or more pluralities of temporal segments {(TS¹₁, TS¹₂, TS¹₃, . . . ), (TS²₁, TS²₂, TS²₃, . . . ), . . . , (TSⁿ₁, TSⁿ₂, TSⁿ₃, . . . )}, such as whether their embeddings into reduced dimensionality space satisfy one or more criteria. For example, in some embodiments, a so-called “population” criterion may be satisfied where at least a threshold number of patients are represented in each cluster of a plurality of clusters detected in the embeddings 108. Another criterion may be whether a so-called “overpopulation” threshold is satisfied—if more than some threshold number of patients are represented in one or more of the clusters, then the cluster(s) may be too populated to be meaningful. As noted above, if a duration of the temporal segments is too long or too short, then the embeddings 108 may tend to clusters that are too populated (e.g., a cluster is not as meaningful if numerous patients with dissimilar clinical histories are included) or not sufficiently populated (e.g., a cluster with too few patients may not provide much evidence of a pattern).

If one or more of the aforementioned criteria are not met when temporal segments of a particular duration are used, then in some embodiments, analysis engine 110 may disregard any patterns observed in embeddings 108 associated with the particular duration. In some embodiments in which pluralities of temporal segments are attempted one duration at a time, if one or more of the aforementioned criteria are not met, analysis engine 110 may notify time chunker 104 that temporal segments of a particular duration are not suitable for embedding, and temporal segments of another duration may be attempted. In some such embodiments, analysis engine 110 may notify time chunker 104 of whether one or more clusters are over or under populated (or whether meaningful clinical trajectories are attainable). Time chunker 104 may then select a new time duration into which to divide the streams of clinical data accordingly.

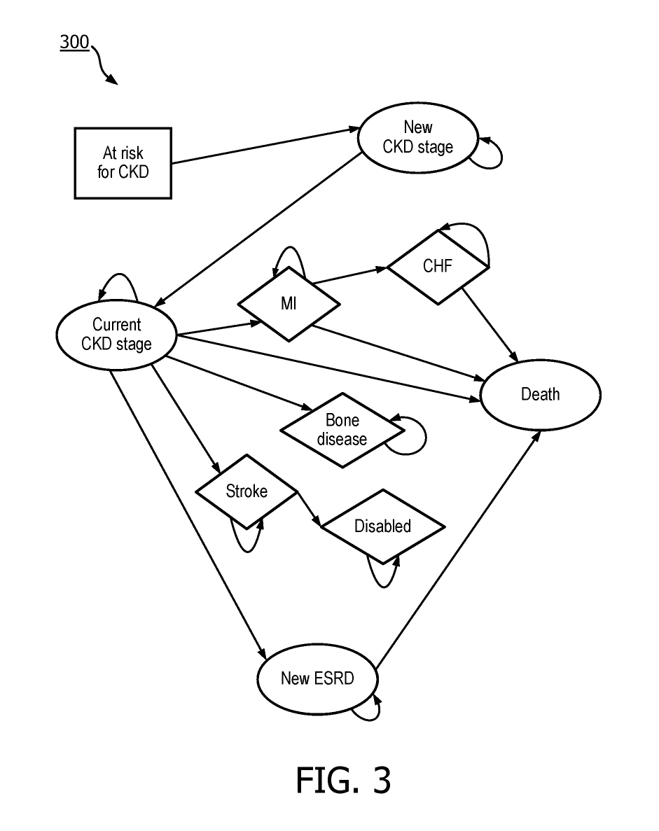

In various embodiments, temporal health trajectories may represent a temporal sequence of flow of clinical events that patients may expect to experience given their clinical past. FIG. 3 depicts one example of a temporal health trajectory associated with chronic kidney disease (“CKD”) that may be gleaned from multiple temporally-connected clusters detected in embeddings 108. As noted above, temporal health trajectories 112 may be used for various purposes.

In some embodiments, temporal health trajectories identified from streams of clinical data associated with a first patient population (e.g., patients of a hospital, a health care system, a state, a country, a county, a clinician pedigree, etc.) may be compared to temporal health trajectories identified from streams of clinical data associated with a second, different patient population. This comparison may reveal, for instance, that patients of the first population tend to experience different temporal health trajectories than patients of the second population. If the temporal health trajectories of the first population are deemed “better” (e.g., higher percentages of positive outcomes, greater avoidance of particular negative outcomes, etc.) than those of the second population, then clinicians, administrators, or other entities that manage health care system(s) of the second population may take appropriate remedial action.

In other embodiments, temporal health trajectories identified from streams of clinical data associated with a patient population may be used to predict/infer a patient's current state, and/or predict and/or infer diagnoses, outcomes, and/or other future clinical events associated with the patient. For example, in some embodiments, the individual's patient's stream of clinical data may be divided, e.g., by time chunker 104, into temporal chunks and embedded, e.g., by embedding engine 106, into a reduced dimensionality space. The patient's individual embeddings may then be matched to existing clusters/trajectories identified by analysis engine 110 previously, e.g., to determine the patient's current state vis-à-vis one or more temporal health trajectories. The next states of the trajectory(ies) and their associated likelihoods or probabilities may then be provided, e.g., by a clinician to the patient, to inform the patient as to what might happen next, and/or to inform the clinician as to what treatments may impact what happens next.

As noted above, in some embodiments, word2vec models may be trained and used to collapse clinical event data into meaningful states of patient care. FIG. 2 depicts a CBOW model on the left and a skip-gram model on the right. These models are often trained using a corpus of textual data to predict either particular words from input surrounding context words (CBOW) or to predict context words (e.g., surrounding words and/or words with similar semantic meaning) from input words (skip-gram). In some embodiments, weights associated with the various layers, such as hidden layers (“PROJECTION” in FIG. 2) and/or output layers, may be initialized as random or other values. Training data may include words and one or more surrounding context words that are applied as input across the models to learn embeddings into a reduced dimensionality space.

In some cases, the CBOW and skip-gram models may be trained end-to-end, as depicted in FIG. 2, similar to encoder/decoder training for neural networks used for image classification. For example, input provided on the left hand side of the CBOW may be forward propagated through the first projection (or hidden) layer (SUM) to reach the first output, w(t), of the CBOW. This output w(t) may then be provided as input to the skip-gram model that is forward propagated to the right-most projection (or hidden) layer, which in turn is further propagated towards the right-hand output layer of the skip-gram model. Because weights associated with the various hidden layers and/or output layers may be initialized to random values, the output of the skip-gram model will be different than the input applied to the CBOW model. This difference, or error, may then be used with techniques such as back propagation and/or stochastic gradient descent to back propagate through the skip-gram and CBOW networks to adjust various weights associated with the various layers. This process may be repeated for the entire input corpus until the models are trained. Thereafter, the models may be used individually to predict context words or words as described above. After training, the weights associated with the hidden (or projection) layer of the skip-gram model may constitute the word embeddings.

In some embodiments of the present disclosure, the skip-gram model may be used, except with temporal segments instead of individual words. That is, each training example used to train the model and learn the embeddings may include a particular temporal segment (which as described above may be an hour, day, week, month, etc.) and any clinical events that occurred during the temporal segment. The training example may also include, as context for the input temporal segment, other temporal segments that surround the input temporal segment (e.g., occur n temporal segments before or after). Accordingly, instead of the trained skip-gram model being able to predict context words (e.g., surrounding words and/or other semantically-related words) based on an input word, the skip-gram model may be used to predict, based on an input temporal segment, other temporal segments that are semantically similar and/or temporally surround the input temporal segment.

If duration(s) of the temporal segments are properly selected, the embeddings may tend to collapse into semantically-similar (or clinically-similar) clusters. In various embodiments, the clusters may be identified in the embeddings using techniques such as hierarchical clustering, centroid-based clustering (e.g., k-means), distribution-based clustering, density-based clustering, and so forth. Additionally, sequences of clusters that tend to follow one another temporally, which are referred to herein as temporal health trajectories, may be identified, e.g., by examining similarities between clusters, examining temporal labels associated with clusters, etc.

FIG. 3 depicts one example of a temporal health trajectory 300 that may be identified using various techniques described above. In FIG. 3, the temporal health trajectory 300 relates to chronic kidney disease (“CKD”). However, this is not meant to be limiting. Temporal health trajectories may be identified for any number of acute and/or chronic conditions, including but not limited to heart disease, diabetes, congestive heart failure, various bodily injuries, pregnancy, liver disease, various cancers, etc. In some embodiments, the various nodes and edges depicted in FIG. 3 may correspond, respectively, to clusters identified in embeddings and relationships (e.g., temporal relationship) between those clusters.

In FIG. 3, the top left node represents a state in which a patient is at risk for CKD. As shown by the single edge, this state may transition to another state in which the patient is officially diagnosed with some new stage of CKD. From there, an edge travels to the patient's current CKD stage, which may lead to several next possible clinical events such as myocardial infarction (“MI”), death, bone disease, stroke, or end-stage renal disease (“ESRD”). While not depicted in FIG. 3, each edge between current state CKD and the next clinical events may have an associated probability or likelihood. These probabilities may be determined, for instance, by examining relationships between the underlying clusters identified in the embeddings. For example, in some embodiments, a probability of one clinical event leading to another may be related to a KL-distance between their respective clusters. In other embodiments, other techniques may be used to identify trajectories between clusters of temporal segments, such as binomial testing (e.g., on a patient-specific, pairwise basis).

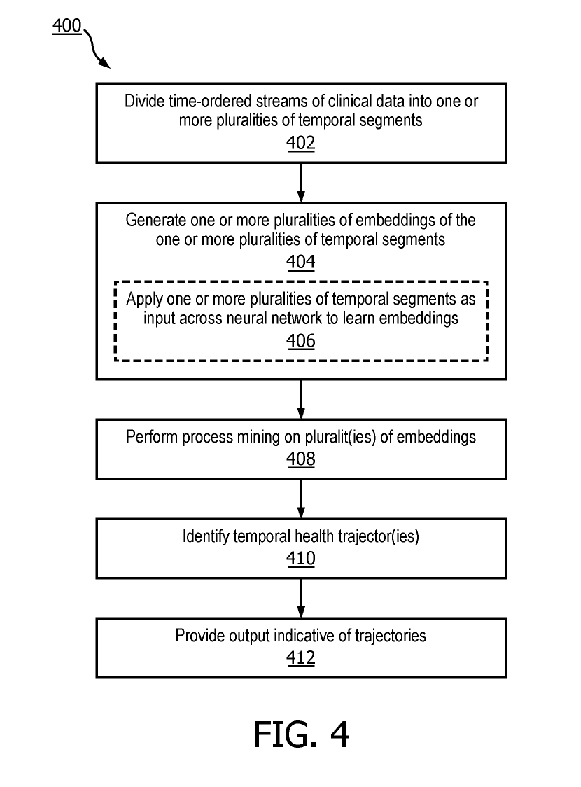

FIG. 4 depicts an example method 400 for practicing selected aspects of the present disclosure, in accordance with various embodiments. For convenience, the operations of the flow chart are described with reference to a system that performs the operations. This system may include various components of various computer systems, including 600. Moreover, while operations of method 400 are shown in a particular order, this is not meant to be limiting. One or more operations may be reordered, omitted or added.

At block 402, the system (e.g., time chunker 104) may divide time-ordered streams of clinical data associated with a plurality of respective patients into one or more respective pluralities of temporal segments. As noted above, each stream of clinical data may indicate, e.g., by way of a sequence of clinical events, a clinical history of a particular patient of the plurality of patients. In some embodiments, each plurality of temporal segments has a different duration. For example, in some embodiments, a first duration may be attempted first to determine whether clusters emerge that satisfy the various population-related criteria described above. If not, then a different duration may be attempted. In other embodiments, multiple durations of temporal segments may be generated at the same time.

At block 404, in some (but not necessarily all) embodiments, the system may generate one or more pluralities of embeddings of the one or more pluralities of temporal segments into a reduced dimensionality space. For example, in some embodiments at optional block 406, the system may applying each plurality of temporal segments created at block 402 as input across a neural network, such as the skip-gram model described above, to learn a respective plurality of embeddings into the reduced dimensionality space. As noted above, with the skip-gram model, the embeddings may be manifested as input weights for the hidden layer of the skip-gram model.

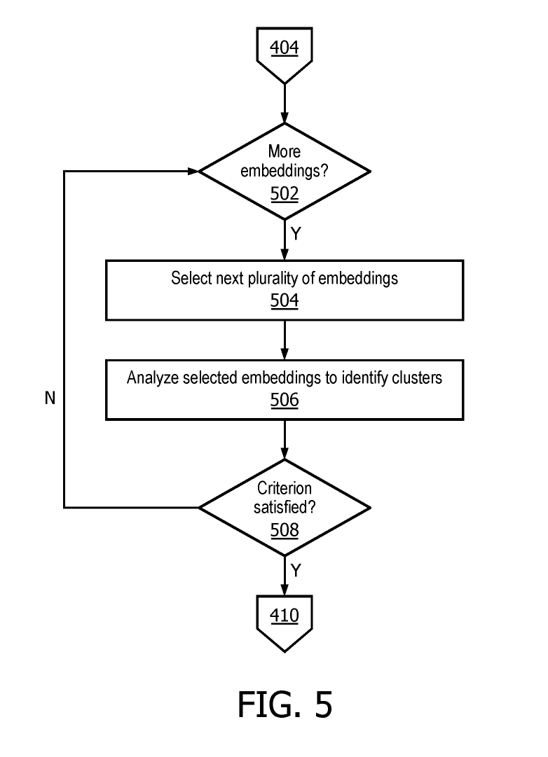

At block 408, the system may perform process mining on the one or more pluralities of embeddings. One example technique for process mining is depicted in FIG. 5. Based on this process mining, at block 410, the system may identify one or more temporal health trajectories shared among the plurality of patients. In some embodiments, this may include generating and/or storing one or more graphs (e.g., directed, undirected, etc.) that represent the temporal health trajectories.

At block 412, the system may output indicative of the temporal health trajectories in various ways. In some embodiments, the temporal health trajectories may be output (or simply stored) as one or more (e.g., directed) graphs that can be used, for instance, to predict one or more clinical events likely to be experienced by patients. For example, in some embodiments a graphical user interface (“GUI”) may be rendered that includes a flowchart that represents a temporal health trajectory, similar to that depicted in FIG. 3. Each node of the flowchart may represent a cluster detected in the embeddings described above. Edges between the nodes may represent temporal transitions between the nodes, and in some cases may include weights that may or may not be included in the GUI as visual renditions. As noted above, in some embodiments these weights may correspond to probabilities or likelihoods of each temporal transition from one node to another. In some embodiments, a user such as a clinician or patient may be able to select (e.g., click, tap) elements of the flowchart to cause additional information to be presented, such as treatment options that might reduce a probability of traversing a given edge, more information (e.g., statistics) about the patients (which may be anonymized) whose data was used to generate the flowchart, and so forth.

In some embodiments in which health care systems are being compared using techniques described herein, multiple flowcharts representing the same or similar health care trajectory may be presented (e.g., side-by-side, simultaneously, overlaid, etc.) for each health care system so that researchers, clinicians, administrators, policy makers, etc., may be able to discern where (and potentially why) outcomes vary between the health care systems. In some embodiments, edges and/or nodes may be visually emphasized (e.g., highlighted, colored conspicuously, animated, annotated, etc.) where they differ from edges/nodes generated from a patient population of another health system. If a particular clinical event is missing in one flowchart (or is at least underrepresented) and that flowchart evidences greater instances of negative outcomes, in some embodiments, the data indicative of the missing clinical event may be presented visually, e.g., as a blinking or dashed line node in the flowchart being considered.

FIG. 5 depicts an example method 500 for practicing selected aspects of the present disclosure, particularly those that occur as part of block 408 (process mining) in FIG. 4, in accordance with various embodiments. For convenience, the operations of the flow chart are described with reference to a system that performs the operations. This system may include various components of various computer systems, including 600. Moreover, while operations of method 500 are shown in a particular order, this is not meant to be limiting. One or more operations may be reordered, omitted or added.

At block 502, which may follow block 404 (and 406 if present) of FIG. 4, the system may determine whether there are more embeddings to analyze. If the answer is yes, then at block 504, the system may select the next plurality of embeddings to analyze. Recall from above that each plurality of embeddings may correspond to (i.e. be generated from) streams of clinical data that are divided into temporal segments of a particular duration. At block 506, the system may analyze the selected plurality of embeddings to identify clusters of temporal segments in the reduced dimensionality space that share one or more attributes. Various cluster identification techniques described previously may be employed.

At block 508, the system may determine whether one or more criteria, such as the population-related criteria described above, are satisfied. Intuitively, the system determines whether the reduced dimensionality embeddings collapse into sufficiently meaningful clusters that can be used to identify temporal health trajectories. If the answer at block 508 is yes, then in some embodiments, control may pass back to block 410 of FIG. 4. If the answer at block 508 is no, then control may pass back to block 502, and the next plurality of embeddings (generated from temporal segments of another duration) may be tested.

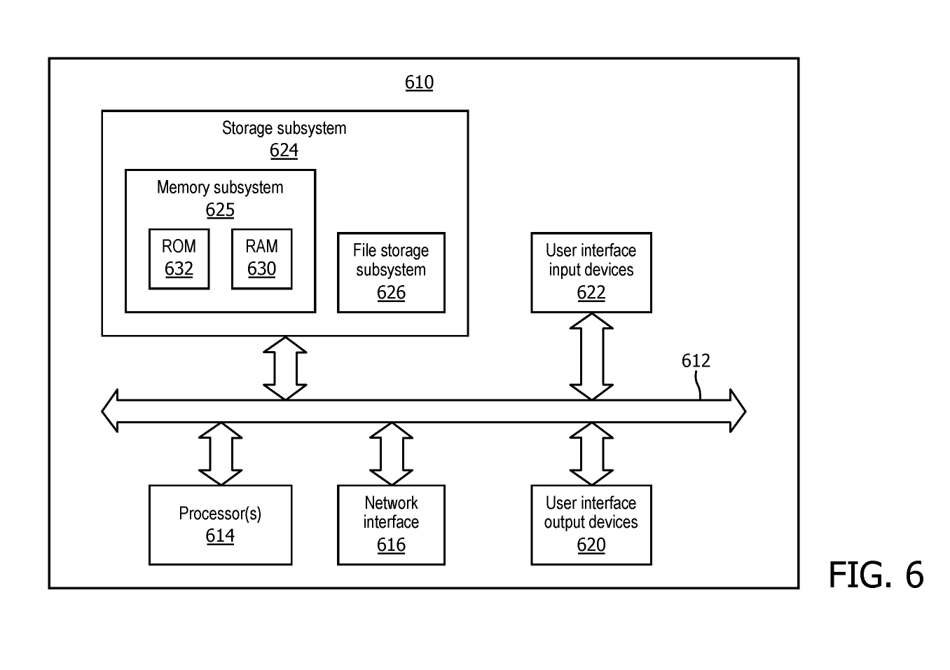

FIG. 6 is a block diagram of an example computer system 610. Computer system 610 typically includes at least one processor 614 which communicates with a number of peripheral devices via bus subsystem 612. As used herein, the term “processor” will be understood to encompass various devices capable of performing the various functionalities attributed to components described herein such as, for example, microprocessors, GPUs, FPGAs, ASICs, other similar devices, and combinations thereof. These peripheral devices may include a data retention subsystem 624, including, for example, a memory subsystem 625 and a file storage subsystem 626, user interface output devices 620, user interface input devices 622, and a network interface subsystem 616. The input and output devices allow user interaction with computer system 610. Network interface subsystem 616 provides an interface to outside networks and is coupled to corresponding interface devices in other computer systems.

User interface input devices 622 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 610 or onto a communication network.

User interface output devices 620 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 610 to the user or to another machine or computer system.

Data retention system 624 stores programming and data constructs that provide the functionality of some or all of the modules described herein. For example, the data retention system 624 may include the logic to perform selected aspects of FIGS. 1-4, as well as to implement selected aspects of methods 400 and/or 500.

These software modules are generally executed by processor 614 alone or in combination with other processors. Memory 625 used in the storage subsystem can include a number of memories including a main random access memory (RAM) 630 for storage of instructions and data during program execution, a read only memory (ROM) 632 in which fixed instructions are stored, and other types of memories such as instruction/data caches (which may additionally or alternatively be integral with at least one processor 614). A file storage subsystem 626 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 626 in the data retention system 624, or in other machines accessible by the processor(s) 614. As used herein, the term “non-transitory computer-readable medium” will be understood to encompass both volatile memory (e.g. DRAM and SRAM) and non-volatile memory (e.g. flash memory, magnetic storage, and optical storage) but to exclude transitory signals.

Bus subsystem 612 provides a mechanism for letting the various components and subsystems of computer system 610 communicate with each other as intended. Although bus subsystem 612 is shown schematically as a single bus, alternative implementations of the bus subsystem may use multiple busses. In some embodiments, particularly where computer system 610 comprises multiple individual computing devices connected via one or more networks, one or more busses could be added and/or replaced with wired or wireless networking connections.

Computer system 610 can be of varying types including a workstation, server, computing cluster, blade server, server farm, or any other data processing system or computing device. In some embodiments, computer system 610 may be implemented within a cloud computing environment. Due to the ever-changing nature of computers and networks, the description of computer system 610 depicted in FIG. 6 is intended only as a specific example for purposes of illustrating some implementations. Many other configurations of computer system 610 are possible having more or fewer components than the computer system depicted in FIG. 6.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

What is claimed is:

1. A method implemented by one or more processors, comprising:
dividing time-ordered streams of clinical data associated with a plurality of respective patients into one or more respective pluralities of temporal segments, wherein each stream of clinical data indicates a clinical history of a particular patient of the plurality of patients, and wherein each of the one or more pluralities of temporal segments has a different duration;
generating one or more pluralities of embeddings of the one or more pluralities of temporal segments into a reduced dimensionality space;
performing process mining on the one or more pluralities of embeddings; and
based on the process mining, identifying one or more temporal health trajectories shared among the plurality of patients.

2. The method of claim 1, wherein the process mining comprises:
analyzing a first plurality of embeddings of the one or more pluralities of embeddings generated from a first plurality of temporal segments having a first duration to identify a first plurality of clusters of temporal segments in the reduced dimensionality space that share one or more attributes;
determining that the first plurality of clusters of temporal segments in the reduced dimensionality space fail to satisfy a population criterion;
analyzing a second plurality of embeddings of the one or more pluralities of embeddings generated from a second plurality of temporal segments having a second duration to identify a second plurality of clusters of temporal segments in the reduced dimensionality space that share one or more attributes; and
determining that the second plurality of clusters of temporal segments in the reduced dimensionality space satisfy the population criterion;
wherein the one or more temporal health trajectories are identified based on the second plurality of clusters of temporal segments.

3. The method of claim 2, wherein the population criterion is satisfied where a threshold number of patients are represented in each of a plurality of clusters.

4. The method of claim 1, wherein the generating comprises applying each of the one or more pluralities of temporal segments as input across a neural network to learn a respective one of the one or more pluralities of embeddings into the reduced dimensionality space.

5. The method of claim 4, wherein the neural network is a skip-gram model.

6. The method of claim 1, wherein each of the one or more pluralities of temporal segments has a duration selected from an hour, a day, a week, or a month.

7. The method of claim 1, wherein each of the one or more pluralities of embeddings is represented as weights associated with a hidden layer of a neural network.

8. The method of claim 1, wherein each temporal segment includes one or more clinical events that occurred during the temporal segment.

9. The method of claim 8, wherein the one or more clinical events are considered coincident within the temporal segment, regardless of an order in which the one or more clinical events actually occurred.

10. At least one non-transitory computer-readable medium comprising instructions that, in response to execution of the instructions by one or more processors, cause the one or more processors to perform the following operations:
dividing time-ordered streams of clinical data associated with a plurality of respective patients into one or more respective pluralities of temporal segments, wherein each stream of clinical data indicates a clinical history of a particular patient of the plurality of patients, and wherein each of the one or more pluralities of temporal segments has a different duration;
generating one or more pluralities of embeddings of the one or more pluralities of temporal segments into a reduced dimensionality space;
performing process mining on the one or more pluralities of embeddings; and
based on the process mining, identifying one or more temporal health trajectories shared among the plurality of patients.

11. The non-transitory computer-readable medium of claim 10, wherein the process mining comprises:
analyzing a first plurality of embeddings of the one or more pluralities of embeddings generated from a first plurality of temporal segments having a first duration to identify a first plurality of clusters of temporal segments in the reduced dimensionality space that share one or more attributes;
determining that the first plurality of clusters of temporal segments in the reduced dimensionality space fail to satisfy a population criterion;
analyzing a second plurality of embeddings of the one or more pluralities of embeddings generated from a second plurality of temporal segments having a second duration to identify a second plurality of clusters of temporal segments in the reduced dimensionality space that share one or more attributes; and
determining that the second plurality of clusters of temporal segments in the reduced dimensionality space satisfy the population criterion;
wherein the one or more temporal health trajectories are identified based on the second plurality of clusters of temporal segments.

12. The non-transitory computer-readable medium of claim 11, wherein the population criterion is satisfied where a threshold number of patients are represented in each of a plurality of clusters.

13. The non-transitory computer-readable medium of claim 10, wherein the generating comprises applying each of the one or more pluralities of temporal segments as input across a neural network to learn a respective one of the one or more pluralities of embeddings into the reduced dimensionality space.

14. The non-transitory computer-readable medium of claim 13, wherein the neural network is a skip-gram model.

15. The non-transitory computer-readable medium of claim 10, wherein each of the one or more pluralities of temporal segments has a duration selected from an hour, a day, a week, or a month.

16. The non-transitory computer-readable medium of claim 10, wherein each of the one or more pluralities of embeddings is represented as weights associated with a hidden layer of a neural network.

17. The non-transitory computer-readable medium of claim 10, wherein each temporal segment includes one or more clinical events that occurred during the temporal segment.

18. The non-transitory computer-readable medium of claim 17, wherein the one or more clinical events are considered coincident within the temporal segment, regardless of an order in which the one or more clinical events actually occurred.

19. A system comprising one or more processors and memory operably coupled with the one or more processors, wherein the memory stores instructions that, in response to execution of the instructions by one or more processors, cause the one or more processors to:
divide time-ordered streams of clinical data associated with a plurality of respective patients into one or more respective pluralities of temporal segments, wherein each stream of clinical data indicates a clinical history of a particular patient of the plurality of patients, and wherein each of the one or more pluralities of temporal segments has a different duration;
generate one or more pluralities of embeddings of the one or more pluralities of temporal segments into a reduced dimensionality space;
perform process mining on the one or more pluralities of embeddings; and
based on the process mining, identify one or more temporal health trajectories shared among the plurality of patients.

20. The system of claim 19, wherein the process mining comprises:
analyzing a first plurality of embeddings of the one or more pluralities of embeddings generated from a first plurality of temporal segments having a first duration to identify a first plurality of clusters of temporal segments in the reduced dimensionality space that share one or more attributes;
determining that the first plurality of clusters of temporal segments in the reduced dimensionality space fail to satisfy a population criterion;
analyzing a second plurality of embeddings of the one or more pluralities of embeddings generated from a second plurality of temporal segments having a second duration to identify a second plurality of clusters of temporal segments in the reduced dimensionality space that share one or more attributes; and
determining that the second plurality of clusters of temporal segments in the reduced dimensionality space satisfy the population criterion;
wherein the one or more temporal health trajectories are identified based on the second plurality of clusters of temporal segments.
